(19) United States
(12) Patent Application Publication
(10) Pub. No.: US 2026/0276649 A1
(43) Pub. Date: Sep. 17, 2026

(54) METHOD FOR EXAMINING FELINE RENAL DYSFUNCTION

(71) Applicant: KAO CORPORATION, Tokyo (JP)

(72) Inventors: Ren KIMURA, Haga-gun (JP); Reeko UEDA, Haga-gun (JP)

(73) Assignee: KAO CORPORATION, Tokyo (JP)

(21) Appl. No.: 19/135,289

(22) PCT Filed: Dec. 20, 2023
(86) PCT No.: PCT/JP2023/045764

§ 371 Date: Jun. 3, 2025

(30) Foreign Application Priority Data
Dec. 21, 2022 (JP) 2022-204807

(51) Int. Cl. G01N 33/68; C12Q 1/26; G01N 33/493

(52) U.S. Cl. CPC G01N 33/6815; C12Q 1/26; G01N 33/493; G01N 2333/90644; G01N 2800/347; G01N 2800/52

(57) ABSTRACT

Provided is an objective and non-invasive method for examining feline renal dysfunction. The method for examining feline renal dysfunction comprises a step of measuring the level of one or more D-amino acids selected from the group consisting of D-tyrosine, D-methionine, D-tryptophane, D-ornithine, and D-histidine in urine of a subject cat.

### FIELD OF THE INVENTION

The present invention relates to examination of feline renal dysfunction using a D-amino acid in urine.

### BACKGROUND OF THE INVENTION

Cats originally evolved in dry areas and therefore have an ability to highly concentrate urine in kidneys and are animals that naturally drink a little water. Accordingly, cats are particularly prone to suffer from urological syndromes such as cystitis, urinary stones, and urethral obstruction due to renal tubular disorders and, furthermore, kidney and urological system diseases such as renal failure. Furthermore, due to recent spread of dry-type food, a tendency of inadequate water intake by cats has been further enhanced. The shortage of water intake increases a risk of causing urological system diseases in the young-aged cats and renal disease in the middle- and old-aged cats and causes a decrease in the quality of life (QOL).

As indicators of the deterioration in feline renal function, the concentration of the serum creatinine and the concentration of the symmetric dimethylarginine (SDMA) are conventionally measured. However, the serum creatinine is believed not to increase until about 75% of the renal function is lost. In contrast, SDMA is believed to begin to increase when 40% of the renal function is lost, but does not necessarily show abnormal values earlier than creatinine, and the reliability thereof as an indicator is not always clear. In addition, it is desired to establish an indicator in urine (non-invasive) to be easily examined and to be on-site monitored, rather than those in blood (minimally invasive) which causes stress and pain.

All amino acids except glycine have two enantiomers, D- and L-isomers. With the improvement in separation ability and sensitivity by recent advance in analytical technology, the existence and the role of D-amino acids in mammals including humans, have been revealed, and it has also been reported that the specific D-amino acid level in blood or urine is associated with the deterioration in the renal function. For example, it has been reported that D-serine and D-alanine in human blood can be used as markers for determining the presence of nephropathy in the critical period (Patent Literatures 1 and 2). In addition, it has been reported that in mice with a decreased renal function, the proportions of D-serine, D-histidine, D-asparagine, D-arginine, D-allo-threonine, D-glutamic acid, D-alanine, D-proline, D-valine, D-allo-isoleucine, D-phenylalanine, and D-lysine in the urine composition are decreased, which can be an indicator for early diagnosis of renal failure (Patent Literature 3). It has also been reported that in dogs and cats with chronic kidney disease, the level of D-valine in urine is significantly decreased (Non Patent Literature 1).
[Patent Literature 1] WO 2020/080494
[Patent Literature 2] WO 2020/080488
[Patent Literature 3] JP-B-5740523
[Non Patent Literature 1] Journal of Pet Animal Nutrition, 20 (the 19th conference issue), 2017, 47-48, Ikeda, et al.

### SUMMARY OF THE INVENTION

The present invention relates to the following 1) to 4):
1) A method for examining feline renal dysfunction, comprising a step of measuring a level of one or more D-amino acids selected from the group consisting of D-tyrosine, D-methionine, D-tryptophane, D-ornithine, and D-histidine in urine of a subject cat;
2) A method for assessing an effect of a preventive or therapeutic intervention for feline renal dysfunction, comprising a step of measuring a level of one or more D-amino acids selected from the group consisting of D-tyrosine, D-methionine, D-tryptophane, D-ornithine, and D-histidine in urine of a subject cat in the presence of the intervention;
3) A method for examining feline renal dysfunction, comprising a step of measuring a total concentration of all D-amino acids that can be measured by an enzyme assay using a D-amino acid oxidase in urine of a subject cat by the assay; and
4) A kit for examining feline renal dysfunction, for carrying out any of the methods of 1) to 3), comprising a reagent for measuring a D-amino acid level.

### BRIEF DESCRIPTION OF DRAWINGS

FIG. 1-1 shows D-amino acid concentrations in feline urine.

FIG. 1-2 shows D-amino acid concentrations in feline urine.

FIG. 2-1 shows L-amino acid concentrations in feline urine.

FIG. 2-2 shows L-amino acid concentrations in feline urine.

FIG. 3-1 shows chiral balances of D-amino acids in feline urine.

FIG. 3-2 shows chiral balances of D-amino acids in feline urine.

FIG. 4 shows total concentrations of D-amino acids and L-amino acids in feline urine.

FIG. 5-1 shows ratios (D/L) of D-amino acid amount to L-amino acid amount in feline urine.

FIG. 5-2 shows ratios (D/L) of D-amino acid amount to L-amino acid amount in feline urine.

FIG. 5-3 shows ratios (D/L) of the total D-amino acid concentration to the total L-amino acid concentration in feline urine.

FIG. 5-4 shows chiral balances of the total D-amino acid concentrations in feline urine.

FIG. 6 shows the total concentration of D-amino acids (Tyr, Met, Trp, Orn, and His) in feline urine.

FIG. 7-1 shows transition of D-amino acids in feline urine.

FIG. 7-2 shows transition of D-amino acids in feline urine.

FIG. 7-3 shows transition of a D-amino acid in feline urine.

FIG. 8-1 shows the total D-amino acid concentrations in feline urine measured by an enzyme assay.

FIG. 8-2 shows chiral balances of the total D-amino acid concentrations in feline urine measured by an enzyme assay.

FIG. 8-3 shows the ratios of the total D-amino acid concentration in feline urine measured by an enzyme assay to urine creatinine.

FIG. 8-4 shows the ratios of the total D-amino acid concentration in feline urine measured by enzyme assay to urine specific gravity.

### DETAILED DESCRIPTION OF THE INVENTION

The present invention relates to providing an objective and non-invasive method for examining feline renal dysfunction.

The present inventors have conducted research in view of such problems and found that in cats with decreased renal functions, the amount of a specific D-amino acid present in urine changes, and assessment of the presence or absence or the degree of feline renal dysfunction or renal function monitoring is possible using the D-amino acid level as an indicator.

According to the method of the present invention, the presence or absence or the degree of feline renal dysfunction can be examined non-invasively, objectively, and easily, and early changes in renal function can be accurately examined.

The method for examining feline renal dysfunction of the present invention has a step of measuring the level of one or more D-amino acids selected from the group consisting of D-tyrosine, D-methionine, D-tryptophane, D-ornithine, and D-histidine in urine of a subject cat.

As used herein, the term “renal dysfunction” refers to a condition in which kidney function is impaired due to some abnormality in the kidney and refers to chronic renal disease that cats are mainly prone to suffer from. The term “chronic renal disease” means a condition in which the kidney function gradually deteriorates to renal failure.

Diagnosis of feline renal dysfunction has conventionally been examined by veterinarian's comprehensive evaluation based on the IRIS (the International Renal Interest Society) Guideline, physical auxiliary examinations (color tone, urine volume, urine specific gravity, and pH), and inquiry. In particular, the degree of renal dysfunction is classified by the IRIS Guideline (2019 modified) as follows:
Healthy: the serum creatinine level is less than 1.6 (mg/dL), or the symmetric dimethylarginine (SDMA) level is less than 14 (μg/dL);
Stage 1 (initial stage): the serum creatinine level is less than 1.6 (mg/dL), or the symmetric dimethylarginine (SDMA) level is from 14 to 17 (μg/dL);
Stage 2 (early stage): the serum creatinine level is from 1.6 to 2.8, or the symmetric dimethylarginine (SDMA) level is from 18 to 25;
Stage 3 (mid stage): the serum creatinine level is from 2.9 to 5.0, or the symmetric dimethylarginine (SDMA) level is 26 to 38; and
Stage 4 (advanced stage): the serum creatinine level is greater than 5.0, or the symmetric dimethylarginine (SDMA) level is greater than 38.

As used herein, the “examination of renal dysfunction” includes examination of the presence or absence of renal dysfunction and the degree of renal dysfunction, preferably the presence or absence of renal dysfunction. The examination of renal dysfunction of the present invention justly includes regular examination and examination for verifying the effect of preventive or therapeutic intervention.

Here, the “examination” can also be rephrased by a term: “measurement”, “detection”, “evaluation”, “assessment”, or “assessment support”.

In the present invention, the subject cat is not particularly limited, and preferably, examples thereof include a cat with suspected renal dysfunction, a cat with a decreased renal function, and a cat subjected to preventive or therapeutic intervention for renal dysfunction. The cat is an animal of the feline family, i.e., an animal of the Family Felidae, Order Carnivora, Mammalia, preferably, a cat belonging to the Subfamily Felidae. The cat is preferably a domestic cat, a grey cat, a jungle cat, an African wildcat, a sand cat, a black-footed cat, or a European wildcat and more preferably a domestic cat (Felis silvestris catus), and the variety thereof is not particularly limited.

The sampling of feline urine that is used as a sample is not particularly limited as long as the urine can be collected under a condition that allows D-amino acid measurement. In terms of measurement accuracy, the urine is preferably fresh. Preferable examples of the method for collecting feline urine include spontaneous urine collection (including use of pet toilet or the like), catheterization, bladder puncture, and pressure urine collection, and spontaneous urine collection which is unlikely to be stressful for cats is particularly preferable.

As shown in Examples below, in feline urine, 20 D-amino acids in total were detected. This is far more than that in human blood (about 4 molecular species) or urine (about 10 molecular species), mouse blood or urine (about 10 molecular species), or a rat blood or urine (about 6 molecular species).

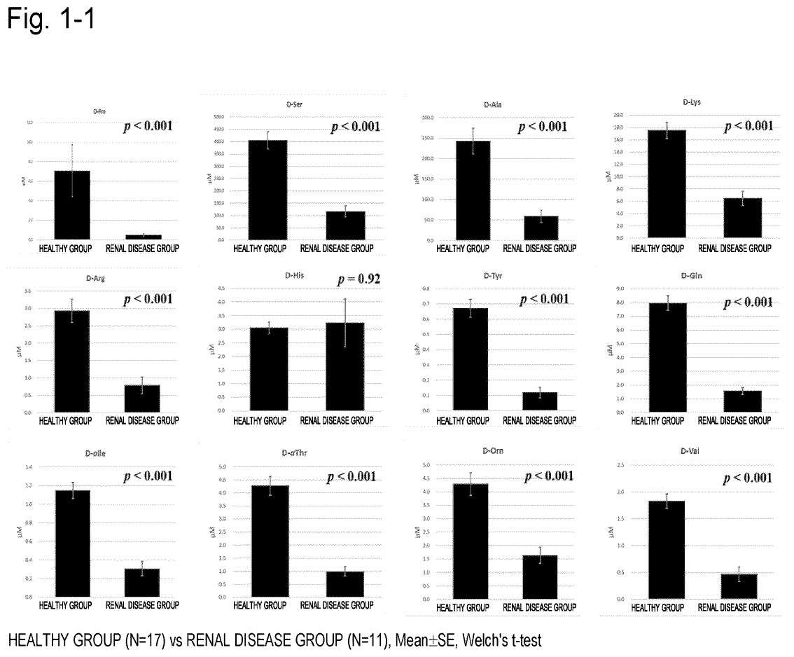

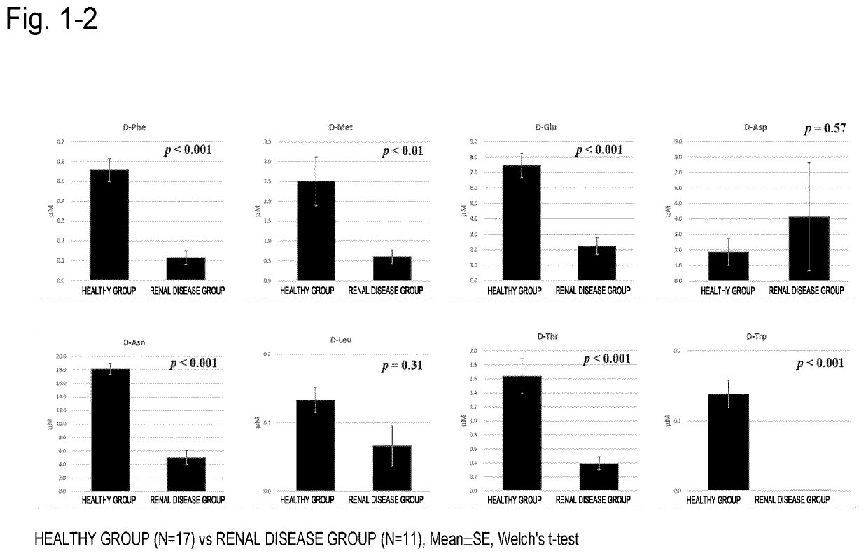

That is, it has been found that the chiral balances of 17 D-amino acids (D-phenylalanine (D-Phe), D-methionine (D-Met), D-glutamic acid (D-Glu), D-leucine (D-Leu), D-threonine (D-Thr), D-tryptophane (D-Trp), D-proline (D-Pro), D-serine (D-Ser), D-alanine (D-Ala), D-lysine (D-Lys), D-arginine (D-Arg), D-tyrosine (D-Tyr), D-glutamine (D-Gin), D-allo-isoleucine (D-aIle), D-allo-threonine (D-aThr), D-ornithine (D-Orn), and D-valine (D-Val)) excluding D-asparagine (D-Asn) and D-aspartic acid (D-Asp) and the quantitative ratio (D/L) of the D-amino acids to their L-amino acids are significantly decreased in the renal disease group and that the chiral balance of D-histidine (D-His) is significantly increased in the renal disease group, unlike the case of mice reported in Patent Literature 3. It is thereby evident that the above is associated with the renal dysfunction (FIGS. 3-1 and 3-2). Among them, D-tyrosine, D-methionine, D-tryptophane, and D-ornithine are amino acids specific to cats, which are notably detected in feline urine but not detected in urine of other mammals such as humans and mice.

Accordingly, D-amino acids composed of in addition to D-histidine, D-tyrosine, D-methionine, D-tryptophane, and D-ornithine in feline urine are marker molecules specific for examining feline renal dysfunction, and the renal dysfunction of a subject cat can be assessed by measuring the existence level of one or more selected from the above D-amino acids.

Although the D-amino acid levels measured in the present invention may be the concentrations of individual D-amino acids selected from the group consisting of D-tyrosine, D-methionine, D-tryptophane, D-ornithine, and D-histidine, preferred are, for example, the quantitative ratio of the D-amino acids to L-amino acids (D-amino acid quantity/L-amino acid quantity) and the chiral balance of the D-amino acids (D-amino acid quantity/(D-amino acid quantity+L-amino acid quantity)×100), and the chiral balance is more preferable.

Regarding the chiral balances of D-amino acids in feline urine, in addition to the measurement of chiral balances of one or more D-amino acids selected from the group consisting of D-tyrosine, D-methionine, D-tryptophane, D-ornithine, and D-histidine, preferably selected from the group consisting of D-tyrosine, D-tryptophane, and D-ornithine, and more preferably selected from the group consisting of D-tyrosine and D-tryptophane, the measurement of chiral balances of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-threonine, D-proline, D-alanine, D-glutamine, D-allo-isoleucine, and D-valine, and more preferably selected from the group consisting of D-phenylalanine, D-glutamic acid, D-alanine, D-allo-isoleucine, and D-valine can be further performed in combination.

Regarding the quantitative ratios (D/L) of D-amino acids to L-amino acids in feline urine, it is preferable to measure the quantitative ratio (D/L) of one or more D-amino acids selected from the group consisting of D-tyrosine, D-tryptophane, D-ornithine, and D-methionine, preferably selected from the group consisting of D-tyrosine, D-tryptophane, and D-ornithine, and more preferably selected from the group consisting of D-tyrosine and D-tryptophane to L-amino acids, and in addition to the above the measurement of quantitative ratios (D/L) of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-proline, D-threonine, D-alanine, D-glutamine, D-allo-isoleucine, and D-valine, and more preferably selected from the group consisting of D-phenylalanine, D-glutamic acid, D-proline, D-alanine, D-allo-isoleucine, and D-valine to L-amino acids can be further performed in combination.

Regarding the D-amino acid concentration in feline urine, it is preferable to measure the concentration of one or more D-amino acids selected from the group consisting of D-tyrosine, D-methionine, D-tryptophane, and D-ornithine and preferably selected from the group consisting of D-tyrosine, D-tryptophane, and D-ornithine, and in addition to the above, the measurement of the concentration of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, D-asparagine, and D-leucine, preferably selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-asparagine, and more preferably selected from the group consisting of D-phenylalanine, D-glutamic acid, D-threonine, D-serine, D-alanine, D-lysine, D-arginine, D-glutamine, D-allo-isoleucine, D-allo-threonine, D-valine, and D-asparagine can be further performed in combination.

Furthermore, the measurement of D-amino acid level in the present invention can also be performed by measuring the total concentration of five D-amino acids consisting of D-tyrosine, D-methionine, D-tryptophane, D-ornithine, and D-histidine, or the total concentration of six or more D-amino acids consisting of the above five D-amino acids and one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid, preferably the total concentration of 20 D-amino acids (D-tyrosine, D-methionine, D-tryptophane, D-ornithine, D-histidine, D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid). Further, the total concentration of D-amino acids including the above-mentioned six or more D-amino acids in feline urine may be the total concentration of all D-amino acids (all D-amino acids excluding acidic amino acids) that can be measured by an enzyme assay using an amino acid as a substrate.

Furthermore, the ratio of the total concentration of the D-amino acids to the total concentration of L-amino acids [(total concentration of D-amino acids)/(total concentration of L-amino acids)] or the chiral balance of the total concentration of the D-amino acids [(total concentration of D-amino acids)/((total concentration of D-amino acids)+(total concentration of L-amino acids))] is calculated, and these values can also be used as the D-amino acid level of the present invention.

Regarding the total concentration of D-amino acids, a ratio of the total concentration of the D-amino acids to urine specific gravity [(total concentration of D-amino acids)/(urine specific gravity)] or a ratio of the total concentration of the D-amino acids to urine creatinine level [(total concentration of D-amino acids)/(urine creatinine level)] is calculated, and these values can also be used as the D-amino acid level of the present invention.

In the present invention, the method for measuring D-amino acids or L-amino acids is not limited as long as D-amino acids and L-amino acids in urine can be separately measured, and generally, a separation and quantification method using LC-MS, LC-MS/MS, or the like, which is a combination of liquid chromatography (LC) and mass spectrometry (MS), can be used. Further, for the measurement of the total amino acid concentration, a quantitative method (enzyme method) utilizing an enzyme reaction (colorimetry, florescence, chemiluminescence, or the like) using a D-amino acid oxidase of which the substrate is a D-amino acid, an L-amino acid oxidase of which the substrate is an L-amino acid, or the like or a biosensing method by a biosensor (electrochemical detection or the like) using an enzyme as an element, can also be use. The enzyme method is more preferable in terms of simplicity and rapidity.

The enzyme assay using a D- or L-amino acid oxidase is a method of detecting or measuring hydrogen peroxide to be generated in oxidative deamination of a D- or L-amino acid by the D- or L-amino acid oxidase with a colorimetric/fluorescent probe, and the total D-amino acid concentration or the total L-amino acid concentration excluding acidic amino acids in feline urine can thereby be measured.

In the measurement of urine amino acids, urine collected at home, in a medical institution, or the like (catheterization, bladder puncture, spontaneous urine collection, and so on) is appropriately applied to pretreatment for measurement, such as freeze and thaw, centrifugation, ultrafiltration, gel filtration, deproteinization, dilution, solid-phase extraction, liquid-liquid distribution, and derivatization. In the pretreatment for the enzyme assay, it is desirable that no inhibitor for the enzyme assay is included. The transportation form of the collected urine is not limited, and may be mailed at from ordinary temperature to frozen.

As the separation method of chiral amino acid, for example, in addition to separation and quantification by changing an amino acid to an NBD derivative using a 4-fluoro-7-nitro-2,1,3-benzoxadiazole (NBD-F) reagent and then performing two-dimensional liquid chromatography (LC) using a reversed phase column (first dimension: molecular species separation) and a chiral column (second dimension: chiral separation) having a chiral identifier-loaded stationary phase (J. Chromatogr. A., 2010, Feb. 12; 1217(7): 1056-62. doi: 10.1016/j.chroma.2009.09.002. Epub, 2009, Sep. 6), one-dimensional LC method using one reversed phase column (ODS column) (Anal. Chim. Acta, 2015, May 22; 875: 73-82. doi: 10.1016/j.aca.2015.02.054. Epub, 2015, Feb. 23), and a one-dimensional LC method by 6-aminoquinolyl-N-hydroxysuccinimidyl carbamate (AQC) derivatization of an amino acid and the use of one chiral column (J. Pharm. Biomed. Anal., 2015, Nov. 10; 115: 123-9. doi: 10.1016/j.jpba.2015.05.024. Epub, 2015, Jun. 16), a method by subjecting an amino group to AQC derivatization and then performing separation by liquid chromatography using a first chiral column including weak anion exchange-type chiral stationary phase and a second chiral column including an amphoteric ion exchange-type chiral stationary phase connected to each other (JP-B-6764778) is known, and the method in JP-B-6764778 is preferable.

Thus, the renal dysfunction of the subject cat can be assessed by comparing the measured D-amino acid level with a reference value.

Here, the reference value can be set, for example, by associating the D-amino acid level with renal dysfunction as follows.

The renal dysfunction is assessed by a means such as the above-described IRIS Guideline. Based on the assessment results, cats are stratified into a healthy group of cats determined to have no renal dysfunction (healthy) and a renal dysfunction group of cats determined to have renal dysfunction. Separately from this, the D-amino acid level of the present invention is measured by the above-described method. Based on correlation between the assessment results of renal dysfunction and the D-amino acid levels, a reference value suitable for assessing renal dysfunction is determined. Specifically, a numerical range of D-amino acid level characterizing each group is determined based on the results of statistical analysis of the D-amino acid level belonging to each group. This numerical range is determined by setting it in a certain range above and below the average value of each group. Here, as the “certain range”, statistical data, such as standard deviation (SD), the ½ SD value, and the ⅓ SD value, may be used, or a certain range above and below the median of each group (such as the first quartile point or the third quartile point) may be used.

For example, when the D-amino acid level of the present invention in urine obtained from a subject cat is within the D-amino acid level range of the renal dysfunction group, the cat can be assessed as “having a decreased renal function”, “having renal dysfunction”, or “having a high risk of renal dysfunction”.

The reference value may be determined based on the cutoff value obtained by statistical analysis.

Examples of the cutoff value include the median or average of the calculated values of biological indicator and values based on ROC curve analysis (for example, Youden's index and distance value from the upper left corner coordinate (0, 1) in an ROC curve created using the discriminant equation for separating groups with and without renal dysfunction). A plurality of cutoff values may be set. For example, it is possible to set respective cutoff values determined by ROC curve analysis as shown in Table 2 below when a chiral balance is the indicator, Table 3 below when the total D-amino acid concentration (5 types or 20 types) is the indicator, Table 4 below when the D-amino acid concentration is the indicator, Table 5 below when the quantitative ratio (D/L) of D-amino acids to L-amino acids is the indicator, and Table 8 below when the total D-amino acid concentration measured by an enzyme assay is the indicator.

For example, it is possible to determine “have a decreased renal function”, “have renal dysfunction”, or “have a high risk of renal dysfunction” when the chiral balance of D-tyrosine is 0.55% or less, the chiral balance of D-methionine is 3.9% or less, the chiral balance of D-tryptophane is 0.10% or less, the chiral balance of D-ornithine is 40% or less, the chiral balance of D-histidine is 2.2% or more, the chiral balance of D-glutamic acid is 4.8% or less, the chiral balance of D-valine is 8.9% or less, the chiral balance of D-phenylalanine is 0.52% or less, the chiral balance of D-allo-isoleucine is 9.8% or less, the chiral balance of D-alanine is 43% or less, the chiral balance of D-proline is 5.5% or less, the chiral balance of D-glutamine is 1.2% or less, the chiral balance of D-threonine is 0.91% or less, the chiral balance of D-serine is 71% or less, the chiral balance of D-allo-threonine is 4.0% or less, the chiral balance of D-arginine is 3.0% or less, the chiral balance of D-lysine is 20% or less, and chiral balance of D-leucine is 0.16% or less.

For example, it is possible to determine “have a decreased renal function”, “have renal dysfunction”, or “have a high risk of renal dysfunction” when the total D-amino acid concentration measured by an enzyme assay is 380.0 μM or less, the chiral balance of the total D-amino acid concentration [(total concentration of D-amino acids measured by enzyme assay/(total concentration of D-amino acids measured by enzyme assay+total concentration of L-amino acids measured by enzyme assay))] is 0.54% or less, the ratio of the total D-amino acid concentration to urine specific gravity is 350.0 μM or less, and the ratio of the total D-amino acid concentration to the urine creatinine level is 1.84 mmol/10 g Cre or less.

Thus, according to the method of the present invention, the presence or the absence of the renal dysfunction in a subject cat and the condition or the degree of the renal dysfunction can be non-invasively, simply, and accurately diagnosed.

Further, the examination method of the present invention may be used in combination with another diagnostic indicator of the renal dysfunction other than the present invention. Consequently, renal dysfunction can be assessed multifacetedly and correctly.

Examples of other diagnostic indicators of the renal dysfunction include blood creatinine (Cre), urine creatinine, blood urea nitrogen (BUN), blood symmetric dimethylarginine (SDMA), blood cystatin C, urine cystatin C, urine neutrophil gelatinase-associated lipocalin (NGAL), urine Liver-type fatty acid binding protein (L-FABP), urine protein, urine protein/creatinine ratio (UPC), and urine albumin. Further, the combination with a physical indicator, such as a simple urine specific gravity assessment method using a specific urine sampling container (JP-B-7084128), urine color (JP-B-7216529), urinary osmolality, fractional excretion rate of various electrolytes, blood pressure, and imaging examination, is also possible.

The examination method of the present invention can be used for detecting the transition in the renal dysfunction of a subject cat by tracking fluctuations in the D-amino acid level through regular implementation.

Further, the examination method of the present invention can be carried out in the presence of the preventive or therapeutic intervention for the feline renal dysfunction for evaluating the effect of the intervention.

That is, the assessment method of the effect of the intervention of the present invention has a step of measuring the level of one or more D-amino acids selected from the group consisting of D-tyrosine, D-methionine, D-tryptophane, D-ornithine, and D-histidine in urine of a subject cat in the presence of a preventive or therapeutic intervention for feline renal dysfunction.

Here, the preventive or therapeutic intervention for feline renal dysfunction is an intervention that is carried out with the expectation of a preventive or therapeutic effect on feline renal dysfunction and encompasses a chemical intervention, a physical or mechanical intervention, and a dietary cure. Examples of the chemical intervention include administration of a substance such as a natural substance, a synthetic substance, and a composition, and examples of the physical or mechanical intervention include treatment such as dialysis. Examples of the dietary cure include feeding of a low-calorie food, a low-fat food, a supplement with adjusted vitamin and mineral content, and a low-salt food.

The effect of the intervention can be assessed based on the D-amino acid level resulted from the intervention in the subject cat. For example, the effect can be assessed by comparing the D-amino acid level in a subject cat in the presence of an intervention with a control (in the absence of the intervention).

A kit for examining feline renal dysfunction of the present invention is a kit for carrying out a method for examining feline renal dysfunction of the present invention. Accordingly, the kit can contain a tool necessary for collecting feline urine, a container for storing feline urine or sand or a sheet containing feline urine, a reagent for measuring the level of one or more D-amino acids, and an indicator or guidance for comparing a D-amino acid level with a reference value.

Examples of the reagent for measuring the D-amino acid level include an enzyme using a D- or L-amino acid as the substrate (e.g., D- or L-amino acid oxidase), a colorimetric or fluorescent probe, and a peroxidase.

Regarding the above-described embodiments, the present invention further discloses the following aspects:
<1> A method for examining feline renal dysfunction, comprising a step of measuring a level of one or more D-amino acids selected from the group consisting of D-tyrosine, D-methionine, D-tryptophane, D-ornithine, and D-histidine in urine of a subject cat;
<2> The method according to <1>, further comprising a step of measuring a level of one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid;
<3> The method according to <1>, wherein the step of measuring the level of D-amino acids is a step of measuring a chiral balance of one or more D-amino acids selected from the group consisting of D-tyrosine, D-methionine, D-tryptophane, D-ornithine, and D-histidine;
<4> The method according to <3>, further comprising a step of measuring a chiral balance of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-threonine, D-proline, D-alanine, D-glutamine, D-allo-isoleucine, and D-valine, and more preferably selected from the group consisting of D-phenylalanine, D-glutamic acid, D-alanine, D-allo-isoleucine, and D-valine;
<5> The method according to <1>, wherein the step of measuring the level of D-amino acids is a step of measuring a quantitative ratio of one or more D-amino acids selected from the group consisting of D-tyrosine, D-tryptophane, D-ornithine, and D-methionine to L-amino acids;
<6> The method according to <5>, further comprising a step of measuring a quantitative ratio of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-proline, D-threonine, D-alanine, D-glutamine, D-allo-isoleucine, and D-valine, and more preferably selected from the group consisting of D-phenylalanine, D-glutamic acid, D-proline, D-alanine, D-allo-isoleucine, and D-valine to L-amino acids;
<7> The method according to <1>, wherein the step of measuring the level of D-amino acids is a step of measuring a concentration of one or more D-amino acids selected from the group consisting of D-tyrosine, D-methionine, D-tryptophane, and D-ornithine;
<8> The method according to <7>, further comprising a step of measuring a concentration of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, D-asparagine, and D-leucine, preferably selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-asparagine, and more preferably selected from the group consisting of D-phenylalanine, D-glutamic acid, D-threonine, D-serine, D-alanine, D-lysine, D-arginine, D-glutamine, D-allo-isoleucine, D-allo-threonine, D-valine, and D-asparagine;
<9> The method according to <1>, wherein the step of measuring the level of D-amino acids comprises a step of measuring a total concentration of five D-amino acids consisting of D-tyrosine, D-methionine, D-tryptophane, D-ornithine, and D-histidine, or a total concentration of six or more D-amino acids consisting of the above-mentioned five D-amino acids and one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid;
<10> The method according to <9>, further comprising a step of calculating a ratio of the total concentration of the D-amino acids to a total concentration of L-amino acids;
<11> The method according to <9>, further comprising a step of calculating a chiral balance of the total concentration of the D-amino acids;
<12> The method according to any one of <9> to <11>, wherein the measurement of the total concentration of six or more D-amino acids is performed by a quantitative method utilizing an enzyme reaction using a D-amino acid as a substrate;
<13> A method for assessing an effect of a preventive or therapeutic intervention for feline renal dysfunction, comprising a step of measuring a level of one or more D-amino acids selected from the group consisting of D-tyrosine, D-methionine, D-tryptophane, D-ornithine, and D-histidine in urine of a subject cat in the presence of the intervention;
<14> The method according to <13>, further comprising a step of measuring a level of one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid;
<15> The method according to <13>, wherein the step of measuring the level of D-amino acids is a step of measuring a chiral balance of one or more D-amino acids selected from the group consisting of D-tyrosine, D-methionine, D-tryptophane, D-ornithine, and D-histidine;
<16> The method according to <15>, further comprising a step of measuring a chiral balance of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-threonine, D-proline, D-alanine, D-glutamine, D-allo-isoleucine, and D-valine, and more preferably selected from the group consisting of D-phenylalanine, D-glutamic acid, D-alanine, D-allo-isoleucine, and D-valine;
<17> The method according to <13>, wherein the step of measuring the level of D-amino acids is a step of measuring a quantitative ratio of one or more D-amino acids selected from the group consisting of D-tyrosine, D-tryptophane, D-ornithine, and D-methionine to L-amino acids;
<18> The method according to <17>, further comprising a step of measuring a quantitative ratio of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine, preferably selected from the group consisting of D-phenylalanine, D-glutamic acid, D-proline, D-threonine, D-alanine, D-glutamine, D-allo-isoleucine, and D-valine, and more preferably selected from the group consisting of D-phenylalanine, D-glutamic acid, D-proline, D-alanine, D-allo-isoleucine, and D-valine to L-amino acids;
<19> The method according to <13>, wherein the step of measuring the level of D-amino acids is a step of measuring a concentration of one or more D-amino acids selected from the group consisting of D-tyrosine, D-methionine, D-tryptophane, and D-ornithine;
<20> The method according to <19>, further comprising a step of measuring a concentration of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, D-asparagine, and D-leucine, preferably selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-asparagine, and more preferably selected from the group consisting of D-phenylalanine, D-glutamic acid, D-threonine, D-serine, D-alanine, D-lysine, D-arginine, D-glutamine, D-allo-isoleucine, D-allo-threonine, D-valine, and D-asparagine;
<21> The method according to <13>, wherein the step of measuring the level of D-amino acids comprises a step of measuring a total concentration of five D-amino acids consisting of D-tyrosine, D-methionine, D-tryptophane, D-ornithine, and D-histidine, or a total concentration of six or more D-amino acids consisting of the above-mentioned five D-amino acids and one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid;
<22> The method according to <21>, further comprising a step of calculating a ratio of the total concentration of the D-amino acids to a total concentration of L-amino acids;
<23> The method according to <21>, further comprising a step of calculating a chiral balance of the total concentration of the D-amino acids;
<24> A method for examining feline renal dysfunction, comprising a step of measuring a total concentration of all D-amino acids which can be measured by an enzyme assay using a D-amino acid oxidase in urine of a subject cat by the assay;
<25> The method according to <24>, further comprising a step of calculating a ratio of the total concentration of the D-amino acids to urine specific gravity;
<26> The method according to <24>, further comprising a step of calculating a ratio of the total concentration of the D-amino acids to a urine creatinine level;
<27> The method according to <24>, further comprising a step of measuring a total concentration of L-amino acids in urine of the subject cat by an enzyme assay using an L-amino acid oxidase;
<28> The method according to <27>, further comprising a step of calculating a chiral balance of the total concentration of the D-amino acids;
<29> The method according to <27>, further comprising a step of calculating a ratio of the total concentration of the D-amino acids to a total concentration of L-amino acids;
<30> A kit for examining feline renal dysfunction, for carrying out any of the methods of <1> to <29>, comprising a reagent for measuring a D-amino acid level;
<31> The kit according to <30>, wherein the reagent for measuring the D-amino acid level comprises a D-amino acid oxidase; and
<32> The kit according to <31>, further comprising a colorimetric or a fluorescent probe and a peroxidase.

### EXAMPLES

The present invention will now be more specifically described with reference to Examples.

### Test Example 1: Simultaneous Separation Analysis of Chiral Amino Acid in Urine of Cat with Kidney Disease

### (1) Regarding Cat Test

The relationship between renal function and urinary components was analyzed for cats (Table 1) in a healthy group (N=17) and a decreased renal function group (N=11, hereinafter, referred to as renal disease group). Urine was collected and was then classified by comprehensive evaluation based on the IRIS (the International Renal Interest Society) Guideline, which is the present standard, physical auxiliary examinations (color tone, urine volume, urine specific gravity, and pH), and veterinarian's inquiry. Classifying into the healthy group and the renal disease group is performed particularly according to the IRIS Guideline, and an individual with a blood (serum) creatinine level of 1.6 (mg/dL) or more or with a symmetric dimethylarginine (SDMA) level of 18 (μg/dL) or more was classified into the renal disease group (corresponding to Stage 2).

TABLE 1

| Classification | Cat species | Sex | Age | Renal disease Stage |
|---|---|---|---|---|
| Renal | Norwegian Forest cat | Contraception ♀ | 13 | Stage 2 (early stage) |
| disease | Scottish Fold | Contraception ♀ | 8 | |
| group | Scottish Fold | Contraception ♀ | 8 | |
| | Catus | ♂ | 18 | |
| | Catus | Contraception ♀ | 16 | |
| | Catus | ♂ | 16 | |
| | Catus | ♂ | 10 | |
| | Catus | ♂ | 4 | |
| | Catus | ♀ | 9 | |
| | Catus | ♂ | 10 | |
| | Siamese | ♂ | 10 | |
| Healthy | MIX | ♂ | 4 | |
| group | Oriental shorthair | ♀ | 4 | |
| | Bengal | ♀ | 1 | |
| | Maine Coon | ♂ | 1 | |
| | American shorthair | ♀ | 1 | |
| | LaPerm | ♂ | 4 | |
| | Ragamuffin | ♀ | 1 | |
| | Napoleon (Minuet) | ♀ | 5 | |
| | Catus | ♂ | 1 | |
| | Catus | ♀ | 2 | |
| | Catus | ♂ | 2 | |
| | MIX | ♂ | 4 | |
| | American curl | Contraception ♀ | 8 | |
| | Chinchilla | Contraception ♀ | 8 | |
| | Russian blue | Castration ♂ | 7 | |
| | Sphynx | ♂ | 5 | |
| | Exotic shorthair | ♂ | 4 | |

### (2) Preparation of Sample Solution and Reference Standard

10 μL of feline urine (spontaneous urine collection) was put in a 10-mL Spitch Glass (trade name, reinforced hard screw-top test tube) and was mixed with 490 μL of a solution of methanol:water (9:1, v/v), and the mixture was then centrifuged with a centrifuge (manufactured by HITACHI, Ltd., CF5RE) at refrigeration (4° C.) for 5 minutes at 2 130×g (3 000 rpm) to remove protein, and the amino acids in the supernatant were collected. Then, separately, a 0.2 mol/L borate buffer (pH 8.9), a collected solution, and an AccQ-Tag Ultra derivatization reagent, i.e., an AQC solution (manufactured by Adipogen Life Sciences, Inc.: a solution prepared by dissolving AQC powder in super-dehydrated acetonitrile in a concentration of 3 mg/mL, i.e., 10 mmol/L) were mixed in this order in the ratio of 7:1:2 (70 μL, 10 μL, and 20 μL, respectively) in a 10-mL Spitch Glass and were immediately stirred, followed by heating at 55° C. for 10 minutes to prepare a derivatized sample solution.

Similarly, 0.2 mol/L borate buffer (pH 8.9), 100 μmol/L D- or L-amino acid standard solution (amino acids: Ala/alanine, Arg/arginine, Asn/asparagine, Asp/aspartic acid, Cys/cysteine, Gln/glutamine, Glu/glutamic acid, Gly/glycine, His/histidine, Ile/isoleucine, allo-Ile/allo-isoleucine, Leu/leucine, Lys/lysine, Met/methionine, Phe/phenylalanine, Pro/proline, Ser/serine, Thr/threonine, allo-Thr/allo-threonine, Trp/tryptophane, Tyr/tyrosine, Val/valine, Cit/citrulline, and Orn/ornithine, 0.2 mol/L borate buffer solution), and the AQC solution were mixed in this order in the ratio of 7:1:2 (70 μL, 10 μL, and 20 μL, respectively), and were immediately stirred, followed by heating at 55° C. for 10 minutes to prepare a derivatized amino acid standard solution.

### (3) LC-MS/MS Analysis

The derivatized sample solution and derivatized amino acid standard solution prepared in (2) were subjected to LC-MS/MS analysis under the following conditions (in accordance with JP-B-6764778) for separation detection and quantification of each chiral amino acid.

### (Apparatus)

Exion LC series (AB Sciex Pte. Ltd.), mass spectrometry/QTRAP 6500⁺ linear ion trap type (AB Sciex Pte. Ltd.).

### (Chromatography Separation)

Separation chiral column: CHIRALPAK QN-AX <Daicel Corporation>,
2.1 mm inner diameter×150 mm, particle diameter of 5 μm (first chiral column) and CHIRALP AK ZWIX(+) <Daicel Corporation>, and
3.0 mm inner diameter×150 mm, particle diameter of 3 μm (second chiral column) serially connected in this order (40° C.);
Eluent: a solution of 0.1% (v/v) formic acid and methanol containing 55 mM ammonium formate:water (90:10, v/v);
Elution method: isocratic;
Mobile phase flow rate: 0.25 mL/min; and
Injection volume: 5 μL.

### (Mass Spectrometry)

Ionization method: electrospray ionization (ESI); and
Polarity: positive ion.
   Curtain Gas (CUR): 30 psi
   Ionspray voltage (IS): 4500 V
   Temperature (TEM): 600° C.
   Ion Source Gas1 (GS1): 80 psi
   Ion Source Gas1 (GS2): 80 psi
   Collision Gas (CAD): 10

### (Detection Mode)

SRM (Selected Reaction Monitoring) detection by the positive ion mode in which a protonated molecule ([M+H]⁺) was set as the precursor ion and an AQC fragment ion (m/z=171) was set as the product ion.

### (4) Data Analysis

### 1) Analysis of Chiral Amino Acid

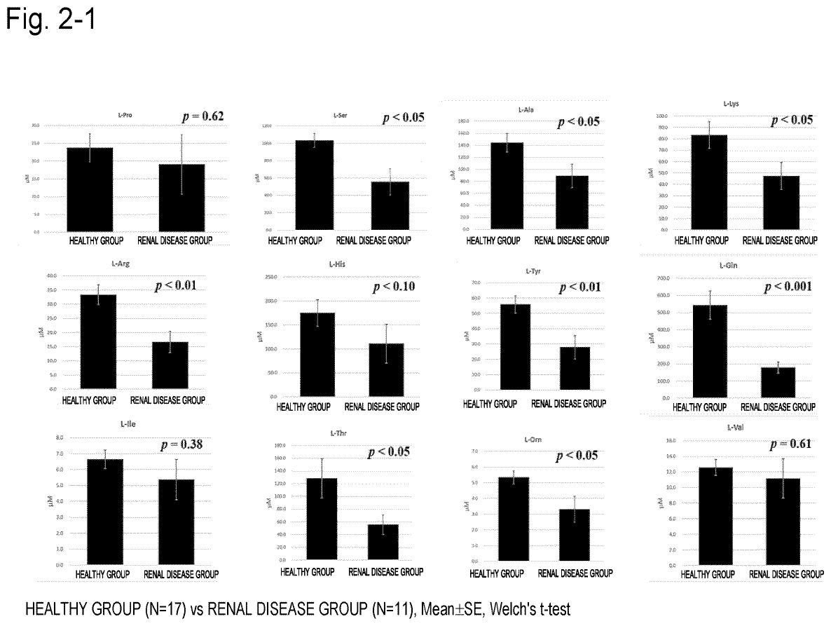

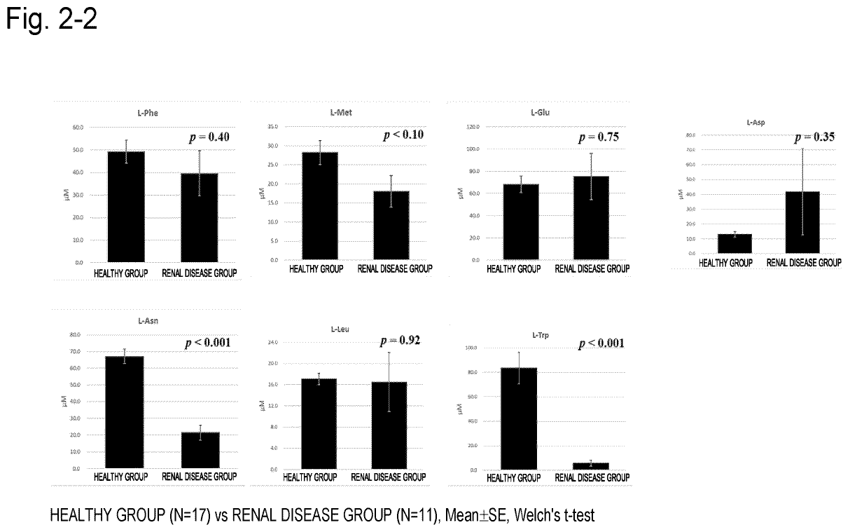

The data obtained in (3) were developed into a chromatogram with two axes: retention time and ion intensity, and AQC derivatized various chiral amino acids in feline urine were analyzed. The urine chiral amino acid concentrations in the healthy group and the renal disease group were measured (The measurement values of D-amino acids (DAA): D-Pro, D-Ser, D-Ala, D-Lys, D-Arg, D-His, D-Tyr, D-Gln, D-allo-Ile (aIle), D-Thr, D-allo-Thr (aThr), D-Orn, D-Val, D-Phe, D-Met, D-Glu, D-Asp, D-Asn, D-Leu, D-Trp are shown in FIGS. 1-1 and 1-2. The measurement values of L-amino acids (LAA): L-Pro, L-Ser, L-Ala, L-Lys, L-Arg, L-His, L-Tyr, L-Gln, L-Ile, L-Thr, L-Orn, L-Val, L-Phe, L-Met, L-Glu, L-Asp, L-Asn, L-Leu, L-Trp are shown in FIGS. 2-1 and 2-2.).

The individual chiral balance (D-isomer abundance ratio: D/(D+L)×100(%)) calculated from the above urine chiral amino acid concentrations are shown in FIGS. 3-1 and 3-2.

It is evident that the chiral balances of 17 amino acids (D-Phe, D-Met, D-Glu, D-Leu, D-Thr, D-Trp, D-Pro, D-Ser, D-Ala, D-Lys, D-Arg, D-Tyr, D-Gln, D-allo-Ile (aIle), D-allo-Thr (aThr), D-Orn, D-Val) excluding D-Asn and D-Asp are significantly decreased in the renal disease group and that the chiral balance of D-His is significantly increased in the renal disease group, and it is confirmed that the chiral balances are associated with the renal function. Among them, D-Tyr, D-Met, D-Trp, and D-Orn are amino acids that are specific to feline urine and are notably detected in feline urine.

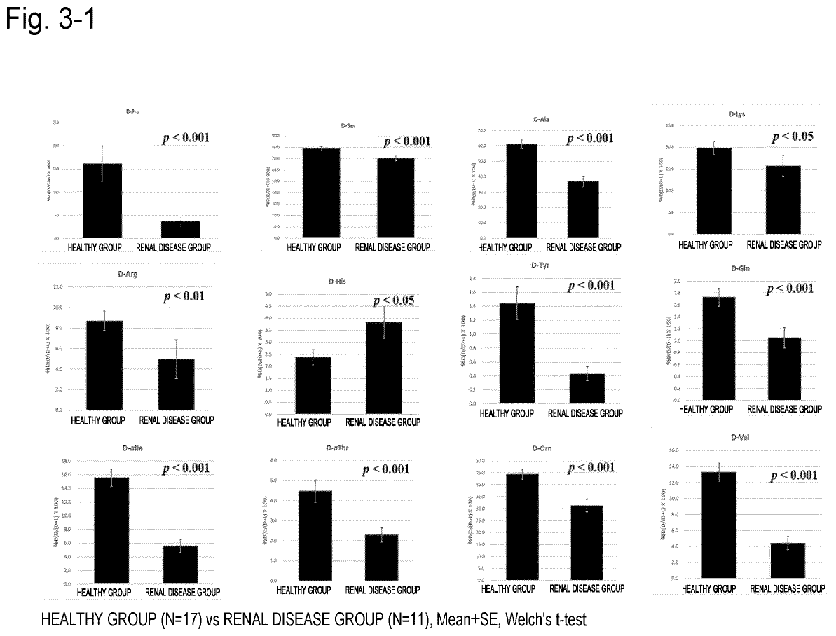

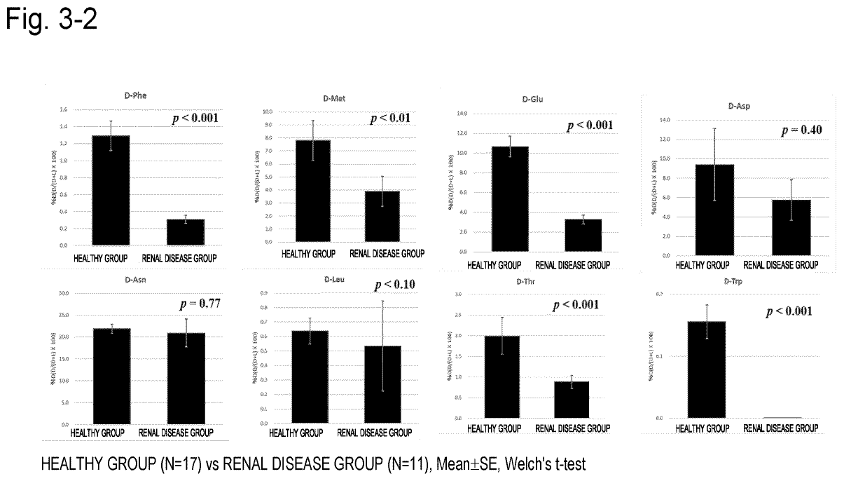

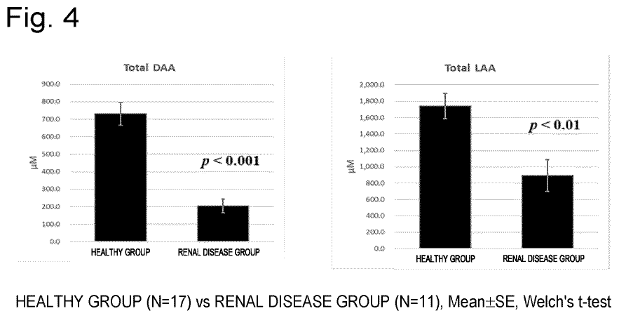

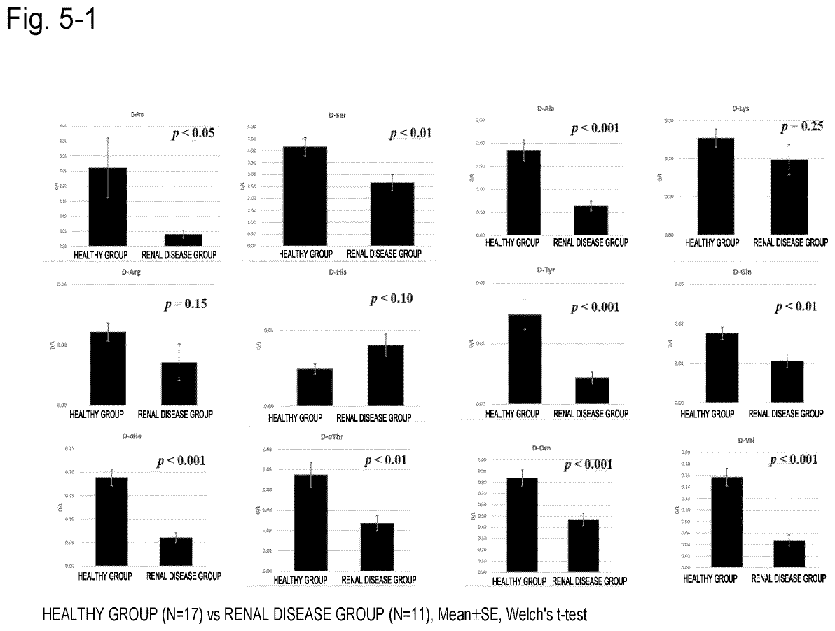

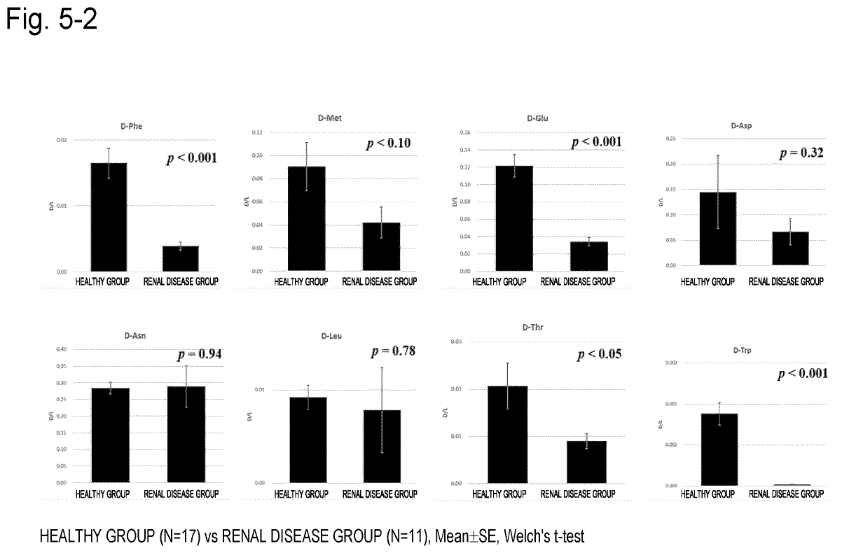

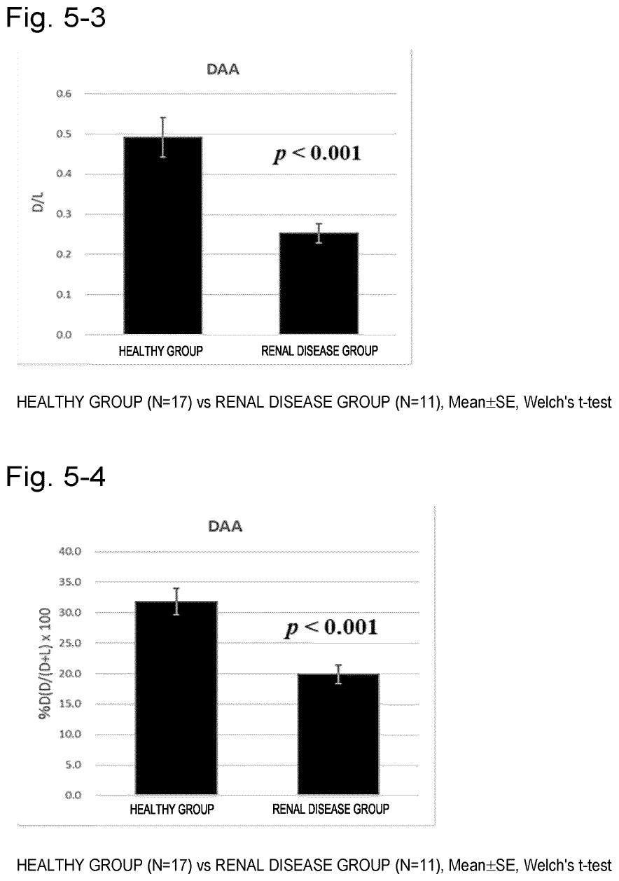

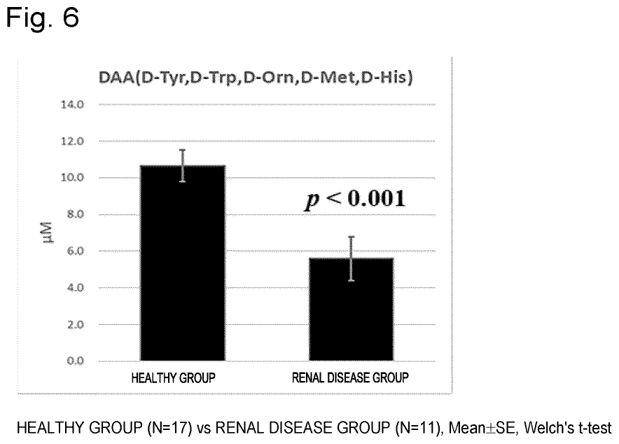

Furthermore, the total DAA or LAA concentrations are shown in FIG. 4, individual D/L ratios are shown in FIGS. 5-1 and 5-2, the D/L ratio of the total concentration is shown in FIG. 5-3, and the chiral balance of the total concentration (D-isomer abundance ratio: D/(D+L)×100(%)) is shown in FIG. 5-4. In addition to the above-described individual chiral balances and concentrations, significant differences are observed between the renal disease group and the healthy group in the quantitative ratios (D/L) of the total concentration and individual DAA and LAA (D-Phe, D-Glu, D-Thr, D-Trp, D-Pro, D-Ser, D-Ala, D-Tyr, D-Gln, D-aIle, D-aThr, D-Orn, D-Val), the significant decrease is also observed in the chiral balance calculated from the total concentration ratio (D/L) of DAA and LAA and total concentration, which indicates it is possible to use as an indicator of renal dysfunction. The total concentration of D-Tyr, D-Met, D-Trp, D-Orn, and D-His only is extracted and shown in FIG. 6. Similarly, the significant decrease is observed in the renal disease group.

### 2) Usefulness Verification

ROC (Receiver Operating Characteristic) curves for the healthy group versus the renal disease group (chiral balance, total concentration, D-amino acid concentration, and quantitative ratio (D/L) of D-amino acid and L-amino acid) were drawn using Excel statistical processing software (BellCurve for Excel, Social survey research information co., ltd.), and cutoff point (Cut off), area under the ROC curve (AUC), sensitivity, specificity, and p value (diagnosability) were each calculated (Table 2: individual chiral balance, Table 3: total concentration, Table 4: D-amino acid concentration, Table 5: quantitative ratio (D/L) of D-amino acid and L-amino acid). In a test in which the Cut off was the closest point to the top left corner and the null hypothesis was AUC=0.50, the significant results were obtained in the chiral balance, (total) concentration, and quantitative ratio (D/L) of D-amino acid and L-amino acid. Thus, diagnosability was observed. A large number of DAA molecular species having an AUC of 0.90 or more was also detected. It is evident that the simple total concentration (DAA (D-Tyr, D-Trp, D-Orn, D-Met, D-His); DAA (Total): D-Pro, D-Ser, D-Ala, D-Lys, D-Arg, D-His, D-Tyr, D-Gln, D-aIle, D-Thr, D-aThr, D-Orn, D-Val, D-Phe, D-Met, D-Glu, D-Asp, D-Asn, D-Leu, D-Trp; LAA (Total): L-Pro, L-Ser, L-Ala, L-Lys, L-Arg, L-His, L-Tyr, L-Gln, L-Ile, L-Thr, L-Orn, L-Val, L-Phe, L-Met, L-Glu, L-Asp, L-Asn, L-Leu, L-Trp) is also useful for the renal function diagnosis, and the usefulness of urine chiral amino acids (in particular, urine DA-A) is recognized.

TABLE 2

| Molecular | Cut off | Sensitivity | Specificity | | p value |
|---|---|---|---|---|---|
| species | (% D) | (%) | (%) | AUC | (diagnosability) |
| D-Tyr | 0.55 | 80 | 100 | 0.930 | p < 0.001 |
| D-Trp | 0.10 | 100 | 82 | 0.912 | p < 0.001 |
| D-Orn | 40.1 | 100 | 71 | 0.882 | p < 0.001 |
| D-Met | 3.95 | 73 | 71 | 0.733 | p < 0.05 |
| D-His | 2.17 | 82 | 53 | 0.701 | p < 0.05 |
| D-Glu | 4.84 | 91 | 100 | 0.979 | p < 0.001 |
| D-Val | 8.88 | 90 | 100 | 0.973 | p < 0.001 |
| D-Phe | 0.52 | 91 | 90 | 0.968 | p < 0.001 |
| D-alle | 9.77 | 85 | 95 | 0.952 | p < 0.001 |
| D-Ala | 43.4 | 80 | 100 | 0.925 | p < 0.001 |
| D-Pro | 5.51 | 82 | 82 | 0.850 | p < 0.001 |
| D-Gln | 1.21 | 73 | 88 | 0.807 | p < 0.001 |
| D-Thr | 0.91 | 82 | 88 | 0.802 | p < 0.001 |
| D-Ser | 70.6 | 64 | 100 | 0.791 | p < 0.001 |
| D-aThr | 3.99 | 100 | 71 | 0.781 | p < 0.01 |
| D-Arg | 2.97 | 64 | 100 | 0.775 | p < 0.01 |
| D-Lys | 19.7 | 91 | 59 | 0.727 | p < 0.05 |
| D-Leu | 0.16 | 73 | 88 | 0.727 | p < 0.05 |
| D-Asn | 17.8 | 45 | 76 | 0.561 | n.s.(nonsignificant) |
| D-Asp | 4.50 | 60 | 47 | 0.503 | n.s.(nonsignificant) |
| Total | 22.0 | 80 | 82 | 0.849 | p < 0.001 |

Healthy group (N = 17) vs Renal disease group (N = 11)

TABLE 3

| | Cut | | | | p value |
|---|---|---|---|---|---|
| Total | off | Sensitivity | Specificity | | (diagnos- |
| concentration | (μM) | (%) | (%) | AUC | ability) |
| DAA | 6.83 | 85 | 80 | 0.870 | p < 0.001 |
| (D-Tyr, D-Trp, | | | | | |
| D-Orn, D-Met, | | | | | |
| D-His) | | | | | |
| DAA (Total) | 420.0 | 100 | 95 | 0.985 | p < 0.001 |
| LAA (Total) | 815.0 | 73 | 100 | 0.845 | p < 0.001 |

Healthy group (N = 17) vs Renal disease group (N = 11)

TABLE 4

| Molecular | Cut off | Sensitivity | Specificity | | p value |
|---|---|---|---|---|---|
| species | (μM) | (%) | (%) | AUC | (diagnosability) |
| D-Tyr | 0.27 | 100 | 91 | 0.989 | p < 0.001 |
| D-Trp | 0.08 | 82 | 100 | 0.904 | p < 0.001 |
| D-Orn | 2.89 | 83 | 100 | 0.909 | p < 0.001 |
| D-Met | 1.60 | 59 | 100 | 0.840 | p < 0.001 |
| D-His | 2.32 | 80 | 50 | 0.626 | n.s. (nonsignificant) |
| D-Glu | 5.16 | 82 | 91 | 0.941 | p < 0.001 |
| D-Val | 0.70 | 100 | 91 | 0.968 | p < 0.001 |
| D-Phe | 0.23 | 100 | 91 | 0.968 | p < 0.001 |
| D-alle | 0.83 | 94 | 100 | 0.968 | p < 0.001 |
| D-Ala | 166.7 | 82 | 100 | 0.963 | p < 0.001 |
| D-Pro | 1.26 | 91 | 76 | 0.882 | p < 0.001 |
| D-Gln | 3.76 | 100 | 100 | 1.000 | p < 0.001 |
| D-Thr | 0.83 | 88 | 91 | 0.963 | p < 0.001 |
| D-Ser | 260.1 | 88 | 100 | 0.973 | p < 0.001 |
| D-aThr | 3.29 | 88 | 100 | 0.957 | p < 0.001 |
| D-Arg | 1.91 | 85 | 100 | 0.925 | p < 0.001 |
| D-Lys | 12.0 | 88 | 100 | 0.973 | p < 0.001 |
| D-Leu | 0.06 | 88 | 73 | 0.727 | p < 0.05 |
| D-Asn | 13.0 | 100 | 100 | 1.000 | p < 0.001 |
| D-Asp | 1.25 | 76 | 34 | 0.519 | n.s. (nonsignificant) |

Healthy group (N = 17) vs Renal disease group (N = 11)

TABLE 5

| Molecular | Cut off | Sensitivity | Specificity | | p value |
|---|---|---|---|---|---|
| species | (D/L) | (%) | (%) | AUC | (diagnosability) |
| D-Tyr | 0.01 | 94 | 82 | 0.930 | p < 0.001 |
| D-Trp | 0.001 | 100 | 100 | 1.000 | p < 0.001 |
| D-Orn | 0.67 | 100 | 71 | 0.882 | p < 0.001 |
| D-Met | 0.04 | 73 | 71 | 0.733 | p < 0.05 |
| D-His | 0.02 | 80 | 53 | 0.700 | n.s. (nonsignificant) |
| D-Glu | 0.07 | 94 | 91 | 0.979 | p < 0.001 |
| D-Val | 0.10 | 82 | 100 | 0.973 | p < 0.001 |
| D-Phe | 0.01 | 88 | 91 | 0.968 | p < 0.001 |
| D-alle | 0.12 | 88 | 100 | 0.952 | p < 0.001 |
| D-Ala | 0.80 | 94 | 82 | 0.925 | p < 0.001 |
| D-Pro | 0.06 | 82 | 82 | 0.850 | p < 0.001 |
| D-Gln | 0.01 | 88 | 73 | 0.807 | p < 0.001 |
| D-Thr | 0.01 | 82 | 88 | 0.802 | p < 0.01 |
| D-Ser | 2.42 | 94 | 64 | 0.791 | p < 0.01 |
| D-aThr | 0.04 | 100 | 71 | 0.781 | p < 0.01 |
| D-Arg | 0.03 | 100 | 64 | 0.775 | p < 0.05 |
| D-Lys | 0.25 | 59 | 91 | 0.727 | p < 0.05 |
| D-Leu | 0.003 | 73 | 88 | 0.727 | p < 0.05 |
| D-Asn | 0.23 | 45 | 76 | 0.561 | n.s. (nonsignificant) |
| D-Asp | 0.06 | 60 | 47 | 0.503 | n.s. (nonsignificant) |
| Total | 0.30 | 80 | 82 | 0.849 | p < 0.001 |

Healthy group (N = 17) vs Renal disease group (N = 11)

### Test Example 2: Time-Dependent Transition of D-Amino Acid in Feline Urine

For cases 1 to 3 shown in Table 6 below, as in Test Example 1, the relationship between renal function and urinary components was analyzed (cat species (1): Abyssinian (castrated male, 3 to 6 years old) was diagnosed with Stage 2 kidney disease through a blood examination in September 2019; cat species (2): Siamese (male, 3 to 4 years) was diagnosed with Stage 2 kidney disease through a blood examination in November 2020; cat species (3): Bengal (castrated male, 4 to 7 years) was diagnosed with Stage 2 kidney disease through a blood examination in November 2016).

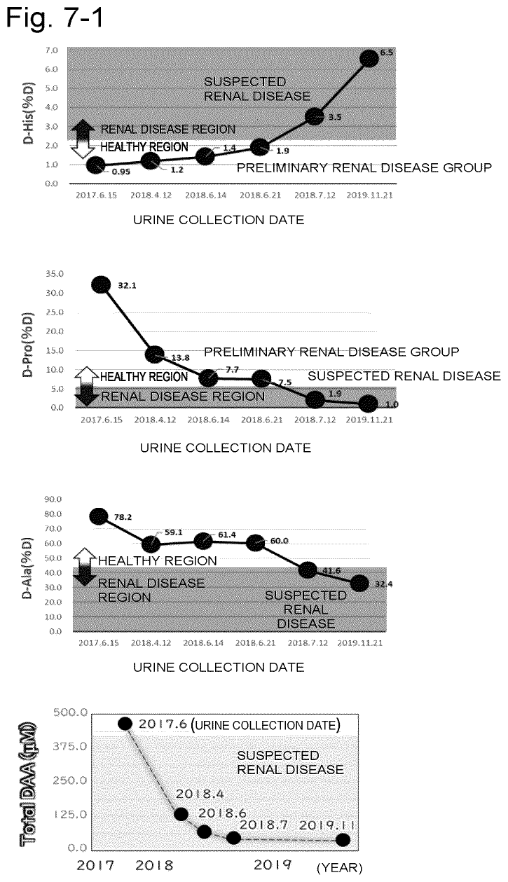

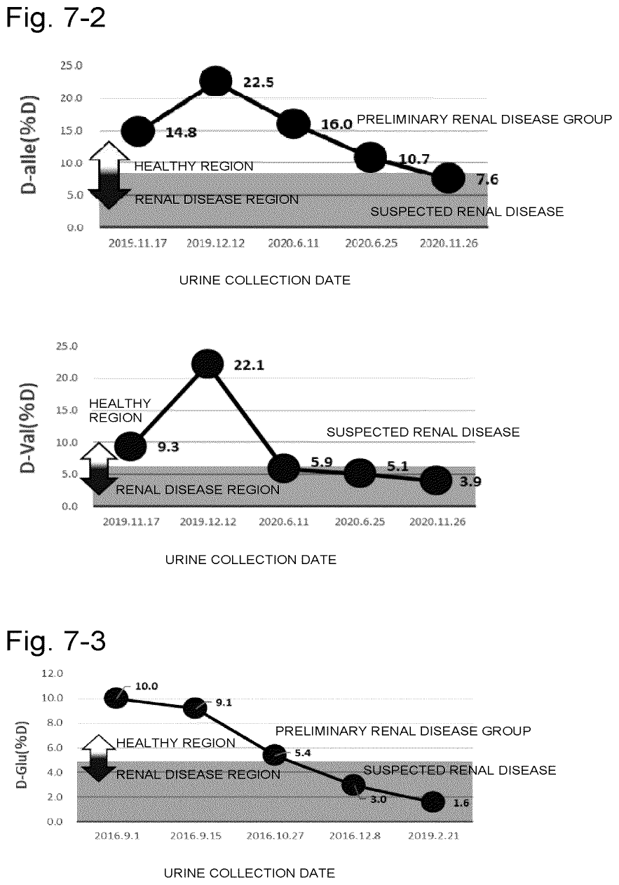

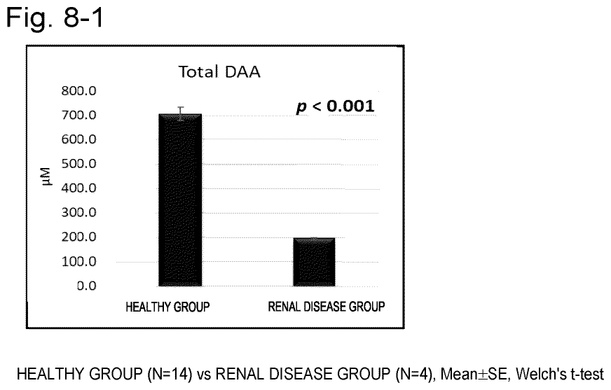

When the time-dependent variations were studied, that is, not only in cross-sectional studies but also in longitudinal studies, the urine chiral balance (for example, D-His, D-aIle, D-Val, and so on) and the urine DAA concentration (total concentration) were recognized to have a relation with a renal function, and were confirmed again to be useful for non-invasive assessment and non-invasive monitoring of the renal function (FIGS. 7-1, 7-2, and 7-3) Urine DAA is an indicator for monitoring the kidney that allows precise assessment of renal severity (disease state) and very early risk for each individual.

TABLE 6

| Cat species | Sex | Age | Renal disease Stage |
|---|---|---|---|
| Abyssinian | Castration ♂ | 3~6 | Stage 2 (2019, September) |
| Siamese | ♂ | 3~4 | Stage 2 (2020, November) |
| Bengal | Castration ♂ | 4~7 | Stage 2 (2016, November) |

### Test Example 3: Analysis of D- and L-Amino Acid in Feline Urine by Enzyme Assay

### (1) Regarding Cat Test

The relationship between renal function and urinary components was analyzed for cats (Table 7) in a healthy group (N=14) and a decreased renal function group (N=4, hereinafter, referred to as renal disease group).

As in Test Example 1, urine was collected and was then classified by comprehensive evaluation based on the IRIS (the International Renal Interest Society) Guideline, which is the present standard, and veterinarian's inquiry. As a feline urine auxiliary examination, the pH (manufactured by HORIBA, Ltd., compact pH meter LAQUAtwin), urine specific gravity (USG) (manufactured by Atago Co., Ltd., PAL-Dog Cat urinometer), and urine creatinine level (CRE) (manufactured by Arkray, Inc., urine chemistry analyzer thinka RT-4010) were measured in combination.

TABLE 7

| | | | | Renal |
|---|---|---|---|---|
| Classi- | | | | disease |
| fication | Cat species | Sex | Age | Stage |
| Renal | Norwegian Forest cat | Contraception ♀ | 13 | Stage 2 |
| disease | Scottish Fold | Contraception ♀ | 8 | (early stage) |
| group | Scottish Fold | Contraception ♀ | 8 | |
| | Siamese | ♂ | 4 | |
| Healthy | MIX | ♂ | 4 | |
| group | Oriental shorthair | ♀ | 4 | |
| | Bengal | ♀ | 1 | |
| | Maine Coon | ♂ | 1 | |
| | American shorthair | ♀ | 1 | |
| | LaPerm | ♂ | 4 | |
| | Ragamuffin | ♀ | 1 | |
| | Napoleon (Minuet) | ♀ | 5 | |
| | MIX | ♂ | 4 | |
| | American curl | Contraception ♀ | 8 | |
| | Chinchilla | Contraception ♀ | 8 | |
| | Russian blue | Castration ♂ | 7 | |
| | Sphynx | ♂ | 5 | |
| | Exotic shorthair | ♂ | 4 | |

### (2) Measurement of Total D-Amino Acid (DAA) Concentration in Feline Urine

### Calibration Curve Creation and Quantitative Value Calculation

The calibration curve creation and quantitative value calculation were performed following the D-Amino Acid Assay Kit (MET-5136, Colorimetric, manufactured by Cell Biolabs, Inc.) protocol. A kit accessory item of the reference standard (D-Alanine) was extemporaneously prepared to draw a calibration curve of 0, 1.56, 3.13, 6.25, 12.5, 25, 50, and 10 μM, and the total D-amino acid concentration (all D-amino acids excluding acidic amino acids) in feline urine was measured.

Specifically, 10 μL of feline urine was placed in a 10-mL Spitch Glass (trade name: reinforced hard screw-top test tube) and was mixed with 90 μL of a kit accessory item of an assay buffer, and the mixture was then centrifuged with a centrifuge (manufactured by HITACHI, Ltd., CF5RE) at refrigeration (4° C.) for 5 minutes at 2 130×g (3 000 rpm) to remove protein, and the amino acids in the supernatant were collected. Subsequently, 50 μL of the above extemporaneously prepared standard solution and 50 μL of feline urine were respectively added to a 96-well microplate (No. 655801, manufactured by Greiner Bio-One). Subsequently, 50 μL of a kit accessory item of an enzyme reaction solution (Colorimetric Probe, HRP, D-Amino Acid Oxidase mixture) was added to each well, and immediately, mixing was performed in the absence of light, followed by incubation at 37° C. for 1 hour (D-amino acid oxidase (DAO) reaction). Finally, the total DAA concentration of the enzyme reaction solution was measured (wavelength: 570 nm) using a microplate reader (SH-9000 Lab, manufactured by Corona Electric Co., Ltd.).

### (3) Measurement of Total L-Amino Acid (LAA) Concentration in Feline Urine

### Calibration Curve Creation and Quantitative Value Calculation

The calibration curve creation and quantitative value calculation were performed following the L-Amino Acid Assay Kit (MET-5154, Colorimetric, manufactured by Cell Biolabs, Inc.) protocol. A kit accessory item of the reference standard (L-Alanine) was extemporaneously prepared to draw a calibration curve of 0, 62.5, 125, 250, 500, and 1 000 μM, and the total L-amino acid concentration in feline urine was measured.

Specifically, 10 μL of feline urine was placed in a 10-mL Spitch Glass (trade name: reinforced hard screw-top test tube) and was mixed with 990 μL of a kit accessory item of an assay buffer, and the mixture was then centrifuged with a centrifuge (manufactured by HITACHI, Ltd., CF5RE) at refrigeration (4° C.) for 5 minutes at 2 130×g (3 000 rpm) to remove protein, and the amino acids in the supernatant were collected. Subsequently, 50 μL of the above extemporaneously prepared standard solution and 50 μL of feline urine were respectively added to a 96-well microplate (No. 655801, manufactured by Greiner Bio-One). Subsequently, 50 μL of a kit accessory item of an enzyme reaction solution (Colorimetric Probe, HRP, L-Amino Acid Oxidase mixture) was added to each well, and immediately, mixing was performed in the absence of light, followed by incubation at 37° C. for 3 hours (L-amino acid oxidase (LAO) reaction).

Finally, the total LAA concentration of the enzyme reaction solution was measured (wavelength: 570 nm) using a microplate reader (SH-9000 Lab, manufactured by Corona Electric Co., Ltd.).

### (4) Data Analysis

### 1) Analysis of DAA-Related Indicator in Feline Urine

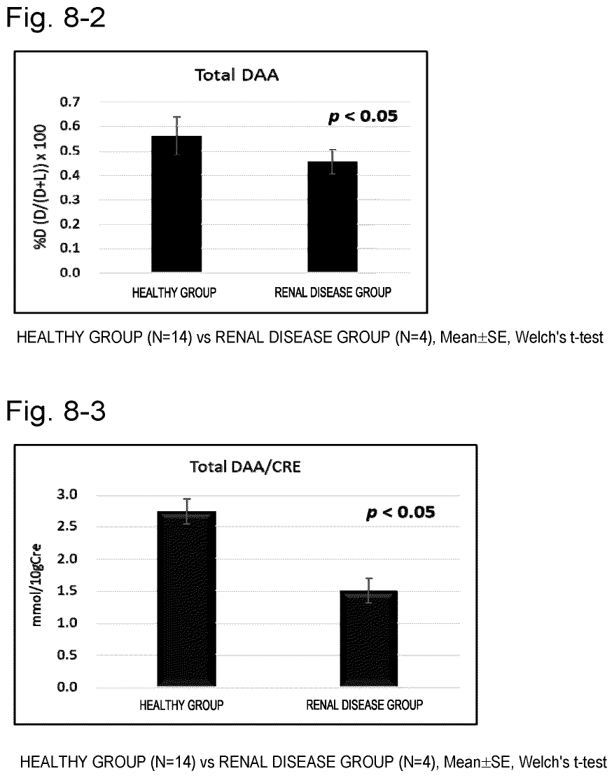

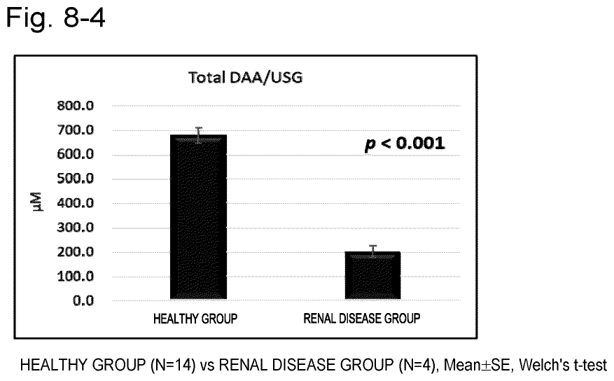

The total DAA concentration measured by the enzyme assay for the healthy group and the renal disease group, the total concentration chiral balance [D-isomer abundance ratio: (total DAA concentration measured by enzyme assay)/((total DAA concentration measured by enzyme assay)+(total LAA concentration measured by enzyme assay))×100(%), the same correlation with the total concentration ratio (total DAA concentration measured by enzyme assay)/(total LAA concentration measured by enzyme assay)], the ratio of (total DAA concentration measured by enzyme assay)/(urine CRE concentration), and the ratio of (total DAA concentration measured by enzyme assay)/USG are shown (FIGS. 8-1, 8-2, 8-3, and 8-4).

### 2) Usefulness Verification

ROC (Receiver Operating Characteristic) curves for the healthy group versus the renal disease group (DAA-related indicator in feline urine) were drawn using Excel statistical processing software (BellCurve for Excel, Social survey research information co., ltd.), and cutoff point (Cut off), area under the ROC curve (AUC), sensitivity, specificity, and p value (diagnosability) were each calculated (Table 8). In a test in which the Cut off was the closest point to the top left corner and the null hypothesis was AUC=0.50, the DAA-related indicator in feline urine was confirmed to have diagnosability. It was found that the total DAA concentration determined by the enzyme assay and its related ratio are particularly useful for renal function assessment.

TABLE 8

| | | | | | p value |
|---|---|---|---|---|---|
| DAA-related | Cut | Sensitivity | Specificity | | (diagnos- |
| indicator | off | (%) | (%) | AUC | ability) |
| Total DAA (μM) | 380.0 | 100 | 100 | 1.000 | p < 0.001 |
| Total DAA/USG | 350.0 | 100 | 100 | 1.000 | p < 0.001 |
| (μM) | | | | | |
| Total DAA (% D) | 0.54 | 100 | 75 | 0.810 | p < 0.05 |
| Total DAA/CRE | 1.84 | 100 | 70 | 0.800 | p < 0.05 |
| (mmol/10 g Cre) | | | | | |

Healthy group (N = 14) vs Renal disease group (N = 4)

1. A method for examining feline renal dysfunction, comprising:
measuring a level of one or more D-amino acids selected from the group consisting of D-tyrosine, D-methionine, D-tryptophane, D-ornithine, and D-histidine in urine of a subject cat.

2. The method according to claim 1, further comprising:
measuring a level of one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid.

3. The method according to claim 1, wherein the measuring the level of D-amino acids comprises measuring a chiral balance of one or more D-amino acids selected from the group consisting of D-tyrosine, D-methionine, D-tryptophane, D-ornithine, and D-histidine.

4. The method according to claim 3, further comprising:
measuring a chiral balance of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine.

5. The method according to claim 1, wherein the measuring the level of D-amino acids comprises measuring a quantitative ratio of one or more D-amino acids selected from the group consisting of D-tyrosine, D-tryptophane, D-ornithine, and D-methionine to L-amino acids.

6. The method according to claim 5, further comprising:
measuring a quantitative ratio of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, and D-leucine to L-amino acids.

7. The method according to claim 1, wherein the measuring the level of D-amino acids comprises measuring a concentration of one or more D-amino acids selected from the group consisting of D-tyrosine, D-methionine, D-tryptophane, and D-ornithine.

8. The method according to claim 7, further comprising:
measuring a concentration of one or more D-amino acids selected from the group consisting of D-phenylalanine, D-glutamic acid, D-threonine, D-proline, D-serine, D-alanine, D-lysine, D-arginine, D-glutamine, D-allo-isoleucine, D-allo-threonine, D-valine, D-asparagine, and D-leucine.

9. The method according to claim 1, wherein measuring the level of D-amino acids comprises measuring a total concentration of five D-amino acids consisting of D-tyrosine, D-methionine, D-tryptophane, D-ornithine, and D-histidine, or a total concentration of six or more D-amino acids consisting of the above-mentioned five D-amino acids and one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid.

10. The method according to claim 9, further comprising:
calculating a ratio of the total concentration of the D-amino acids to a total concentration of L-amino acids.

11. The method according to claim 9, further comprising:
calculating a chiral balance of the total concentration of the D-amino acids.

12. A method for assessing an effect of a preventive or therapeutic intervention for feline renal dysfunction, the method comprising:
measuring a level of one or more D-amino acids selected from the group consisting of D-tyrosine, D-methionine, D-tryptophane, D-ornithine, and D-histidine in urine of a subject cat in the presence of the intervention.

13. The method according to claim 12, wherein the measuring the level of D-amino acids comprises measuring a chiral balance of one or more D-amino acids selected from the group consisting of D-tyrosine, D-methionine, D-tryptophane, D-ornithine, and D-histidine.

14. The method according to claim 12, wherein the measuring the level of D-amino acids comprises measuring a quantitative ratio of one or more D-amino acids selected from the group consisting of D-tyrosine, D-tryptophane, D-ornithine, and D-methionine to L-amino acids.

15. The method according to claim 12, wherein the measuring the level of D-amino acids comprises measuring a concentration of one or more D-amino acids selected from the group consisting of D-tyrosine, D-methionine, D-tryptophane, and D-ornithine.

16. The method according to claim 12, wherein the measuring the level of D-amino acids comprises measuring a total concentration of five D-amino acids consisting of D-tyrosine, D-methionine, D-tryptophane, D-ornithine, and D-histidine, or a total concentration of six or more D-amino acids consisting of the above-mentioned five D-amino acids and one or more D-amino acids selected from the group consisting of D-phenylalanine, D-glutamic acid, D-leucine, D-threonine, D-proline, D-serine, D-alanine, D-lysine, D-arginine, D-glutamine, D-allo-isoleucine, D-allo-threonine, D-valine, D-asparagine, and D-aspartic acid.

17. The method according to claim 16, further comprising:
calculating a ratio of the total concentration of the D-amino acids to a total concentration of L-amino acids.

18. The method according to claim 16, further comprising:
calculating a chiral balance of the total concentration of the D-amino acids.

19. A method for examining feline renal dysfunction, comprising:
measuring a total concentration of all D-amino acids that can be measured by an enzyme assay using a D-amino acid oxidase in urine of a subject cat by the assay.

20. The method according to claim 19, further comprising:
calculating a ratio of the total concentration of the D-amino acids to urine specific gravity.

21. The method according to claim 19, further comprising:
calculating a ratio of the total concentration of the D-amino acids to a urine creatinine concentration.

22. The method according to claim 19, further comprising:
measuring a total concentration of all L-amino acids that can be measured by an enzyme assay using an L-amino acid oxidase in the urine of the subject cat.

23. The method according to claim 22, further comprising:
calculating a chiral balance of the total concentration of the D-amino acids.

24. The method according to claim 22, further comprising:
calculating a ratio of the total concentration of the D-amino acids to a total concentration of L-amino acids.

25. A kit for examining feline renal dysfunction, suitable for carrying out the method of claim 1, the kit comprising:
a reagent for measuring a D-amino acid level.

26. The kit according to claim 25, wherein the reagent for measuring the D-amino acid level comprises a D-amino acid oxidase.

27. The kit according to claim 26, further comprising:
a colorimetric or a fluorescent probe and a peroxidase.
